(19) United States
(12) Patent Application Publication
(10) Pub. No.: US 2007/0128125 A1
(43) Pub. Date: Jun. 7, 2007

(54) Pharmaceutical Compositions Based on Tiotropium Salts and Salts of Salmeterol

(75) Inventors: Christel Schmelzer, Ingelheim (DE); Juergen Nagel, Bad Kreuznach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma KG, Ingelheim (DE)

(21) Appl. No.: 11/537,333

(22) Filed: Sep. 29, 2006

Related U.S. Application Data
(63) Continuation of application No. 10/054,567, filed on Nov. 13, 2001, now abandoned.
(60) Provisional application No. 60/251,603, filed on Dec. 6, 2000.

(30) Foreign Application Priority Data
Nov. 13, 2000 (DE) 100 56 104

(51) Int. Cl. A61K 31/4745; A61K 31/138; A61K 9/14

(52) U.S. Cl. 424/46; 514/291; 514/651

(57) ABSTRACT

A pharmaceutical composition, comprising: (a) a tiotropium salt (1); and (b) a salmeterol salt (2), optionally in the form of the enantiomers, mixtures of enantiomers, or in the form of the racemates thereof, optionally in the form of the solvates or hydrates and optionally together with a pharmaceutically acceptable excipient, and methods of treating respiratory diseases using such a pharmaceutical composition.

### RELATED APPLICATIONS

This application is a continuation of U.S. patent application Ser. No. 10/054,567, filed Nov. 13, 2001, which claims priority to U.S. Ser. No. 60/251,603, filed Dec. 6, 2000, the contents of which are hereby incorporated by reference in their entirety.

### FIELD OF THE INVENTION

The present invention relates to novel pharmaceutical compositions based on tiotropium salts and salts of salmeterol, processes for preparing them and their use in the treatment of respiratory complaints.

### BACKGROUND OF THE INVENTION

The compound tiotropium bromide, a salt of tiotropium, is known from European Patent Application EP 418 716 A1 and has the following chemical structure

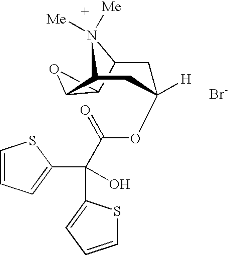

This compound may also be referred to by its chemical name (1α,2β,4β,5α,7β)-7-[(hydroxydi-2-thienylacetyl)oxy]-9,9-dimethyl-3-oxa-9-azoniatricyclo[3.3.1.0^{2,4}]nonane bromide and has valuable pharmacological properties. The name tiotropium is intended to refer to the free cation for the purposes of the present invention.

Like other salts of tiotropium, it is a highly effective anticholinergic and can therefore provide therapeutic benefit in the treatment of asthma or COPD (chronic obstructive pulmonary disease).

Tiotropium salts are preferably administered by inhalation. Suitable inhalable powders packed into appropriate capsules (inhalettes) and administered using suitable powder inhalers may be used. Alternatively, they may be administered by the use of suitable inhalable aerosols. These also include powdered inhalable aerosols which contain, for example, HFA134a, HFA227 or mixtures thereof as propellant gas. The tiotropium salts may also be inhaled in the form of suitable solutions.

### BRIEF DESCRIPTION OF THE DRAWINGS

FIG. 1A: shows a longitudinal section through the atomiser with the spring under tension.

FIG. 1B: shows a longitudinal section through the atomiser with the spring released.

### DETAILED DESCRIPTION OF THE INVENTION

Surprisingly, it has been found that an unexpectedly beneficial therapeutic effect, more particularly a synergistic effect, in the treatment of inflammatory or obstructive respiratory complaints is observed when one or more tiotropium salts (1) are used in conjunction with one or more salmeterol salts (2).

As a result, it is possible to reduce substantially the undesirable side effects which often occur, for example, in the administration of β-mimetics such as salmeterol to humans. Examples of the central side effects of β-mimetics include general malaise, excitation, sleeplessness, anxiety, trembling fingers, sweats and headaches.

The name tiotropium is intended to refer to the free cation (1′) for the purposes of the present invention. References to salmeterol are intended as references to the free base (2′) for the purposes of the present invention.

The combinations of active substances according to the invention are surprisingly also characterised by a rapid onset of activity and also by long-lasting effects. This is of great importance to the well being of the patient as, on the one hand, they experience a rapid improvement in their condition after administration of the combination and, on the other hand, a single administration per day is sufficient, thanks to the long-lasting effect.

The abovementioned effects are observed both after the simultaneous administration within a single active substance formulation and also after the successive administration of the two active substances in separate formulations. It is preferred according to the invention to administer the two active substance ingredients simultaneously in a single formulation.

In one aspect the present invention relates to a pharmaceutical composition which contains one or more tiotropium salts (1) and one or more salmeterol salts (2), optionally in the form of the solvates or hydrates thereof. The active substances may be contained either together in a single preparation or in two separate preparations. According to the invention, pharmaceutical compositions which contain the active substances 1 and 2 in a single preparation are preferred.

According to another aspect the present invention relates to a pharmaceutical composition which contains, in addition to therapeutically effective amounts of 1 and 2 a pharmaceutically acceptable excipient. In one aspect the present invention relates to a pharmaceutical composition which contains, in addition to therapeutically effective amounts of 1 and 2 no pharmaceutically acceptable excipient.

The present invention further relates to the use of 1 and 2 for preparing a pharmaceutical composition containing therapeutically effective amounts of 1 and 2 for the treatment of inflammatory or obstructive respiratory complaints, particularly asthma or COPD, by simultaneous or successive administration.

The present invention is also directed to the simultaneous or successive use of therapeutically effective doses of the combination of the abovementioned pharmaceutical compositions 1 and 2 for the treatment of inflammatory or obstructive respiratory complaints, particularly asthma or COPD.

The tiotropium salts 1 which may be used within the scope of the present invention include the compounds which contain, in addition to tiotropium, chloride, bromide, iodide, methanesulphonate, para-toluenesulphonate or methylsulphate as counter-ion (anion). Within the scope of the present invention, of all the tiotropium salts, the methanesulphonate, chloride, bromide or iodide are preferred, the methanesulphonate or bromide being of particular importance. Tiotropium bromide is of exceptional importance according to the invention.

By salts of salmeterol 2 are meant, according to the invention, pharmaceutically acceptable salts selected from among the salts of hydrochloric acid, hydrobromic acid, sulphuric acid, phosphoric acid, methanesulphonic acid, acetic acid, fumaric acid, succinic acid, lactic acid, citric acid, tartaric acid, xinafonic acid or maleic acid, with the proviso that 2 cannot denote salmeterol xinafoate if 1 denotes tiotropium bromide. Mixtures of the abovementioned acids may optionally also be used to prepare the salmeterol salts.

According to the invention, the salmeterol salts 2 selected from among hydrochloride, hydrobromide, sulphate, phosphate and methanesulphonate are preferred. The salts of 2 selected from hydrochloride and sulphate are particularly preferred, especially the sulphates. Salmeterol×½H₂SO₄ is of exceptional importance according to the invention.

In the combinations of active substances according to the invention consisting of 1 and 2 the ingredients 1 and 2 may be in the form of their enantiomers, mixtures of enantiomers or in the form of the racemates.

The proportions in which the two active substances 1 and 2 may be used in the combinations of active substances according to the invention are variable. The active substances 1 and 2 may optionally be in the form of the solvates or hydrates thereof. Depending on the choice of the salts 1 or 2 the weight ratios which may be used for the purposes of the present invention vary on account of the different molecular weights of the various salt forms. Consequently, the weight ratios specified hereinafter are based on the tiotropium cation 1′ and the free base of salmeterol 2′. The combinations of active substances according to the invention may contain 1′ and 2′ in weight ratios in the range from 1:300 to 30:1, preferably from 1:230 to 20:1, particularly preferably from 1:150 to 10:1, more preferably from 1:50 to 5:1, particularly preferably from 1:35 to 2:1. Of particular interest according to the invention are pharmaceutical compositions containing the combination of 1′ and 2′ in a weight ratio in the range from 1:25 to 1:1, preferably in the range from 1:10 to 1:2, particularly preferably in the range from 1:5 to 1:2.5.

For example, without restricting the scope of the invention, preferred combinations of 1 and 2 according to the invention may contain tiotropium 1′ and salmeterol 2′ in the following weight ratios: 1:40; 1:20; 1:11.1; 1:10; 1:5.6; 1:5; 1:2.8; 1:2.5; 1:1.4; 1:1.25; 1.44:1, 1.6:1.

The pharmaceutical compositions according to the invention containing the combinations of 1 and 2 are normally used so that tiotropium 1′ and salmeterol 2′ are administered together in doses of 0.01 to 10000 μg, preferably 0.1 to 2000 μg, particularly preferably from 1 to 1000 μg, more preferably from 5 to 500 μg, preferably, according to the invention, from 10 to 200 μg, preferably from 20 to 100 μg, most preferably from 30 to 70 μg per single dose.

For example, combinations of 1 and 2 according to the invention contain an amount of tiotropium 1′ and salmeterol 2′ such that the total dosage per single dose is 30 μg, 35 μg, 45 μg, 55 μg, 60 μg, 65 μg, 90 μg, 105 μg, 110 μg, 110 μg, 140 μg or similar. In these dosage ranges the active substances 1′ and 2′ are present in the weight rations described hereinbefore.

For example, without restricting the scope of the invention, the combinations of 1 and 2 according to the invention may contain an amount of tiotropium 1′ and salmeterol 2′ such that 5 μg of 1′ and 25 μg of 2′, 5 μg of 1′ and 50 μg of 2′, 5 μg of 1′ and 100 μg of 2′, 5 μg of 1′ and 200 μg of 2′, 10 μg of 1′ and 25 μg of 2′, 10 μg of 1′ and 50 μg of 2′, 10 μg of 1′ and 100 μg of 2′, 10 μg of 1′ and 200 μg of 2′, 18 μg of 1′ and 25 μg of 2′, 18 μg of 1′ and 50 μg of 2′, 18 μg of 1′ and 100 μg of 2′, 18 μg of 1′ and 200 μg of 2′, 20 μg of 1′ and 25 μg of 2′, 20 μg of 1′ and 50 μg of 2′, 20 μg of 1′ and 100 μg of 2′, 20 μg of 1′ and 200 μg of 2′, 36 μg of 1′ and 25 μg of 2′, 36 μg of 1′ and 50 μg of 2′, 36 μg of 1′ and 100 μg of 2′, 36 μg of 1′ and 200 μg of 2′, 40 μg of 1′ and 25 μg of 2′, 40 μg of 1′ and 50 μg of 2′, 40 μg of 1′ and 100 μg of 2′ or 40 μg of 1′ and 200 μg of 2′ are administered per single dose.

If the active substance combination in which 1 denotes tiotropium bromide and 2 denotes salmeterol×½H₂SO₄ is used as the preferred combination of 1 and 2 according to the invention, the quantities of active substances 1′ and 2′ administered per single dose as specified above by way of example correspond to the following quantities of 1 and 2 administered per single dose: 6 μg of 1 and 27.9 μg of 2, 6 μg of 1 and 55.9 μg of 2, 6 μg of 1 and 111.8 μg of 2, 6 μg of 1 and 223.6 μg of 2, 12 μg of 1 and 27.9 μg of 2, 12 μg of 1 and 55.9 μg of 2, 12 μg of 1 and 111.8 μg of 2, 12 μg of 1 and 223.6 μg of 2, 21.7 μg of 1 and 27.9 μg of 2, 21.7 μg of 1 and 55.9 μg of 2, 21.7 μg of 1 and 111.8 μg of 2, 21.7 μg of 1 and 223.6 μg of 2, 24.1 μg of 1 and 27.9 μg of 2, 24.1 μg of 1 and 55.9 μg of 2, 24.1 μg of 1 and 111.8 μg of 2, 24.1 μg of 1 and 223.6 μg of 2, 43.3 μg of 1 and 27.9 μg of 2, 43.3 μg of 1 and 55.9 μg of 2, 43.3 μg of 1 and 111.8 μg of 2, 43.3 μg of 1 and 223.6 μg of 2, 48.1 μg of 1 and 27.9 μg of 2, 48.1 μg of 1 and 55.9 μg of 2, 48.1 μg of 1 and 111.8 μg of 2 or 48.1 μg of 1 and 223.6 μg of 2.

If, in the combination of 1 and 2 preferred according to the invention, wherein 2 denotes salmeterol×½ H₂SO₄ tiotropium bromide monohydrate is used as 1 for example, the quantities of active substances 1′ and 2′ administered per single dose as specified above by way of example correspond to the following quantities of 1 and 2 administered per single dose: 6.2 μg of 1 and 27.9 μg of 2, 6.2 μg of 1 and 55.9 μg of 2, 6.2 μg of 1 and 111.8 μg of 2, 6.2 μg of 1 and 223.6 μg of 2, 12.5 μg of 1 and 27.9 μg of 2, 12.5 μg of 1 and 55.9 μg of 2, 12.5 μg of 1 and 111.8 μg of 2, 12.5 μg of 1 and 223.6 μg of 2, 22.5 μg of 1 and 27.9 μg of 2, 22.5 μg of 1 and 55.9 μg of 2, 22.5 μg of 1 and 111.8 μg of 2, 22.5 μg of 1 and 223.6 μg of 2, 25 μg of 1 and 27.9 μg of 2, 25 μg of 1 and 55.9 μg of 2, 25 μg of 1 and 111.8 μg of 2, 25 μg of 1 and 223.6 μg of 2, 45 μg of 1 and 27.9 μg of 2, 45 μg of 1 and 55.9 μg of 2, 45 μg of 1 and 111.8 μg of 2, 45 μg of 1 and 223.6 μg of 2, 50 μg of 1 and 27.9 μg of 2, 50 μg of 1 and 55.9 μg of 2, 50 μg of 1 and 111.8 μg of 2 or 50 μg of 1 and 223.6 μg of 2.

The combinations of active substances 1 and 2 according to the invention are preferably administered by inhalation. For this purpose, the ingredients 1 and 2 have to be incorporated in inhalable preparations.

Suitable inhalable preparations include inhalable powders, metering aerosols containing propellant gases or inhalable solutions free from propellant gases. Inhalable powders according to the invention containing the active substance combination of 1 and 2 may consist solely of the abovementioned active substances or of a mixture of the abovementioned active substances with physiologically acceptable adjuvants. Within the scope of the present invention the term propellant-free solutions for inhalation also includes concentrates or sterile, ready-to-use solutions for inhalation. The preparations according to the invention may contain the active substance combination of 1 and 2 either together in one preparation or in two separate preparations. These preparations which may be used within the scope of the present invention are described in detail in the following section of the specification.

A) Inhalable Powders Containing the Active Substance Combinations of 1 and 2 According to the Invention:

The powders for inhalation according to the invention may contain 1 and 2 either on their own or in admixture with suitable physiologically harmless adjuvants.

If the active substances 1 and 2 are present in admixture with physiologically harmless adjuvants, the following physiologically harmless adjuvants may be used to prepare these inhalable powders according to the invention: monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these adjuvants with one another. Mono- or disaccharides are preferably used, the use of lactose or glucose, particularly but not exclusively in the form of their hydrates, being preferred. The particularly preferred adjuvant according to the invention is lactose, most preferably lactose monohydrate.

Within the scope of the powders for inhalation according to the invention the adjuvants have a maximum mean particle size of up to 250 μm, preferably between 10 and 150 μm, particularly preferably between 15 and 80 μm. If desired it may be useful to add finer adjuvant fractions having a mean particle size of 1 to 9 μm to the abovementioned adjuvants. These latter finer adjuvants are also selected from the abovementioned group of adjuvants which may be used. Finally, in order to prepare the powders for inhalation according to the invention, micronised active substance 1 and 2, preferably having an average particle size of 0.5 to 10 μm, particularly preferably from 1 to 6 μm, is added to the adjuvant mixture. Processes for preparing the powders for inhalation according to the invention by grinding and micronising and finally mixing the ingredients together are known from the prior art. The powders for inhalation according to the invention may be prepared and administered either in the form of a single powder mixture which contains both 1 and 2, or in the form of separate inhalable powders which contain only 1 and 2.

The inhalable powders according to the invention can be administered using inhalers known from the prior art.

Inhalable powders according to the invention which contain a physiologically harmless adjuvant in addition to 1 and 2 may for example be administered using inhalers which meter a single dose from a reservoir by means of a measuring chamber, as described in U.S. Pat. No. 4,570,630A, or by other devices as described in DE 36 25 685 A. Preferably, the inhalable powders according to the invention which contain physiologically harmless adjuvant in addition to 1 and 2 are packed into capsules (to form so-called inhalettes), which are used in inhalers such as those described, for example, in WO 94/28958. If the inhalable powders according to the invention are to be packed into capsules (inhalettes) as in the preferred application mentioned above, fillings of 1 to 30 mg, preferably from 3 to 20 mg, preferably 5 to 10 mg of inhalable powder per capsule are suggested. According to the invention, these contain the dosages specified above for 1′ and 2′ either together or separately per single dose.

B) Inhalable Aerosols Containing Propellant, Comprising the Active Substance Combinations of 1 and 2 According to the Invention:

Inhalable aerosols containing propellant according to the invention may contain 1 and 2 dissolved in the propellent gas or in dispersed form. 1 and 2 may be present in separate preparations or in a combined preparation, with 1 and 2 either both dissolved, both dispersed or only one component dissolved while the other is present in dispersed form.

The propellent gases which can be used to prepare the inhalable aerosols according to the invention are known from the prior art. Suitable propellent gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellent gases may be used on their own or in mixtures thereof. Particularly preferred propellent gases are halogenated alkane derivatives selected from among TG11, TG12, TG134a and TG227. Of the abovementioned halogenated hydrocarbons, TG 134a (1,1,1,2-tetrafluoroethane) and TG227 (1,1,1,2,3,3,3-heptafluoropropane) and mixtures thereof are preferred according to the invention.

The propellant-gas-containing inhalable aerosols according to the invention may also contain other ingredients such as cosolvents, stabilisers, surface-active agents (surfactants), antioxidants, lubricants and means for adjusting the pH. All these ingredients are known in the art.

The propellant-gas-containing inhalable aerosols according to the invention may contain up to 5% by weight of active substance 1 and/or 2. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active substance 1 and/or 2.

If the active substances 1 and/or 2 are present in dispersed form the particles of active substance preferably have a mean particle size of up to 10 μm, preferably from 0.1 to 5 μm, particularly preferably from 1 to 5 μm.

The abovementioned propellant-gas-containing inhalable aerosols according to the invention can be administered by means of inhalers known in the art (MDIs=metered dose inhalers). Accordingly, a further aspect of the present invention relates to pharmaceutical compositions in the form of propellant-gas-containing aerosols as described above combined with one or more inhalers suitable for administering these aerosols. Furthermore, the present invention relates to inhalers, characterised in that they contain the propellant-gas-containing aerosols according to the invention as described above.

The present invention also relates to cartridges which are fitted with a suitable valve and can be used in a suitable inhaler and which contain one of the abovementioned propellant-gas-containing inhalable aerosols according to the invention. Suitable cartridges and methods of filling these cartridges with the propellant-gas-containing inhalable aerosols according to the invention are known from the prior art.

C) Propellant-Free Inhalable Solutions Containing the Active Substance Combinations of 1 and 2 According to the Invention:

It is particularly preferable for the active substance combination according to the invention to be administered in the form of propellant-free solutions for inhalation. Suitable solvents for this include aqueous or alcoholic, preferably ethanolic solutions. The solvent may be water on its own or a mixture of water and ethanol. The relative proportion of ethanol to water is not restricted, but the maximum limit is preferably up to 70 percent by volume, particularly up to 60 percent by volume and most preferably up to 30 percent by volume. The remaining percent by volume are made up with water. The preferred solvent is water without the addition of ethanol. The solutions containing 1 and 2, separately or together, are adjusted to a pH of 2 to 7, preferably 2 to 5, particularly preferably 2.5 to 3.5, with suitable acids. Most preferably, inhalable solutions according to the invention which contain 1 and 2 together have a pH of about 2.9. This pH may be achieved using acids selected from among inorganic or organic acids. Examples of particularly suitable inorganic acids include hydrochloric acid, hydrobromic acid, nitric acid, sulphuric acid and/or phosphoric acid. Examples of particularly suitable organic acids include: ascorbic acid, citric acid, malic acid, tartaric acid, maleic acid, succinic acid, fumaric acid, acetic acid, formic acid and/or propionic acid and others. Preferred inorganic acids are hydrochloric acid and sulphuric acid. It is also possible to use the acids which are forming an acid addition salt with the active substance or, in the case of combined preparations, with one of the active substances. Of the organic acids, ascorbic acid, fumaric acid and citric acid are preferred. If desired, mixtures of the abovementioned acids may also be used, particularly in the case of acids which have properties other than their acidifying properties, e.g. as flavourings, antioxidants or complexing agents, such as citric acid or ascorbic acid, for example. According to the invention, it is particularly preferred to use hydrochloric acid to adjust the pH.

According to the invention, there is no need to add editic acid (EDTA) or one of the known salts thereof, sodium edetate, to the present formulation as a stabiliser or complexing agent. Other embodiments contain these compound(s).

In a preferred embodiment of this kind, the content based on sodium edetate is less than 100 mg/100 ml, preferably less than 50 mg/100 ml, most preferably less than 20 mg/100 ml. Inhalable solutions in which the content of sodium edetate is 0 to 10 mg/100 ml are generally preferred.

Co-solvents and/or other adjuvants may be added to the propellant-free inhalable solutions according to the invention. Preferred co-solvents are those which contain hydroxyl groups or other polar groups, e.g. alcohols—particularly isopropylalcohol, glycols—particularly propyleneglycol, polyethyleneglycol, polypropyleneglycol, glycolether, glycerol, polyoxyethylene alcohols and polyoxyethylene fatty acid esters. By excipients and additives is meant, in this context, any pharmacologically acceptable substance which is not an active substance, but can be formulated together with the active substance(s) in the pharmacologically suitable solvent, in order to improve the qualitative properties of the active substance formulation. Preferably, these substances do not have any appreciable pharmacological effects or at least have no undesirable effects in the context of the intended therapy. The excipients and additives include e.g. surfactants such as e.g. soya lecithin, oleic acid, sorbitan esters such as polysorbates, polyvinylpyrrolidone, other stabilisers, complexing agents, antioxidants and/or preservatives which guarantee or extend the shelf life of the finished pharmaceutical formulation, flavourings, vitamins and/or other additives known in the art. The additives also include pharmacologically harmless salts such as sodium chloride, for example, as isotonic agents.

The preferred adjuvants include antioxidants, such as ascorbic acid, for example, unless it has already been used to adjust the pH, vitamin A, vitamin E, tocopherols and similar vitamins or provitamins occurring in the human body.

Preservatives can be used to protect the formulation from contamination with pathogens. Suitable preservatives are those known from the prior art, particularly cetylpyridinium chloride, benzalkonium chloride or benzoic acid or benzoates such as sodium benzoate in the concentration known from the prior art. The above-mentioned preservatives are preferably present in concentrations of up to 50 mg/100 ml, particularly between 5 and 20 mg/100 ml.

Preferred formulations contain only benzalkonium chloride and sodium edetate in addition to the solvent water and the active substance combination of 1 and 2. In another preferred embodiment, sodium edetate is omitted.

The propellant-free inhalable solutions according to the invention may be administered particularly using inhalers which are able to nebulise a small amount of a liquid formulation in the therapeutically necessary dose within a few seconds to form an aerosol suitable for therapeutic inhalation. Within the scope of the present invention, nebulisers are preferred in which a quantity of less than 100 μL, preferably less than 50 μL, particularly preferably between 20 and 30 μL of active substance solution can be nebulised, preferably in one operation, to produce an aerosol having an average particle size of less than 20 μm, preferably less than 10 μm, in such a way that the inhalable part of the aerosol corresponds to the therapeutically effective amount.

A device of this kind for the propellant-free administration of a metered amount of a liquid pharmaceutical composition for inhalation is described in detail, for example, in International Patent Application WO 91/14468 and also in WO 97/12687 (particularly FIGS. 6a and 6b). The nebulisers (devices) described therein are also known by the name Respimat®.

This nebuliser (Respimat®) can advantageously be used to produce inhalable aerosols according to the invention containing the active substance combination of 1 and 2. Because of its cylindrical shape and handy size of less than 9 to 15 cm long and 2 to 4 cm wide this device can be carried anywhere by the patient. The nebuliser sprays a defined volume of the pharmaceutical formulation under high pressure through small nozzles, so as to produce inhalable aerosols.

The preferred atomiser essentially consists of an upper housing part, a pump housing, a nozzle, a locking clamp, a spring housing, a spring and a storage container, characterised by
a pump housing which is fixed in the upper housing part and carries, at one end, a nozzle member with the nozzle or nozzle arrangement,
a hollow piston with valve member,
a power take-off flange in which the hollow piston is secured, and which is located in the upper housing part,
a locking clamp which is located in the upper housing part,
a spring housing with the spring located therein, which is rotatably mounted on the upper housing part by means of a rotary bearing,
a lower housing part which is fitted onto the spring housing in the axial direction.

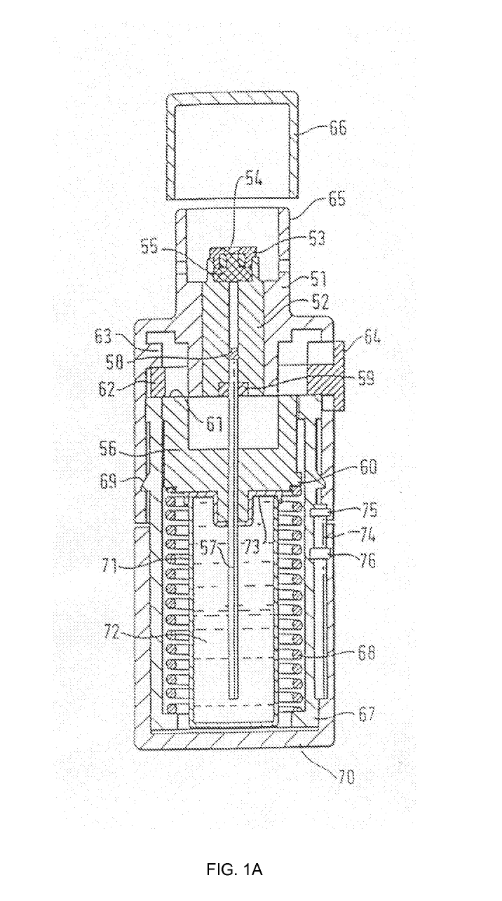

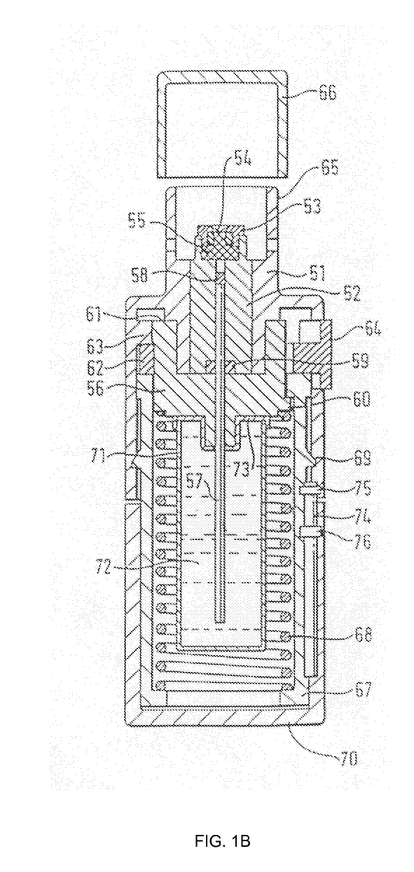

The hollow piston with valve member corresponds to a device disclosed in WO 97/12687. It projects partially into the cylinder of the pump housing and is mounted to be axially movable in the cylinder. Reference is made particularly to FIGS. 1-4-—particularly FIG. 3—and the associated parts of the description. At the moment of actuation of the spring, the hollow piston with valve member exerts a pressure at its high pressure end of 5 to 60 Mpa (about 50 to 600 bar), preferably 10 to 60 Mpa (about 100 to 600 bar) on the fluid, the measured quantity of active substance solution. Volumes of 10 to 50 microlitres are preferred, volumes of 10 to 20 microlitres are particularly preferred, while a volume of 15 microlitres per spray is most particularly preferred.

The valve member is preferably mounted at the end of the hollow piston facing the nozzle member.

The nozzle in the nozzle member is preferably microstructured, i.e. produced by microtechnology. Microstructured nozzle members are disclosed, for example, in WO-94/07607; reference is hereby made to this specification, particularly FIG. 1 and the description thereof.

The nozzle member consists e.g. of two sheets of glass and/or silicon firmly joined together, at least one of which has one or more microstructured channels which connect the nozzle inlet end to the nozzle outlet end. At the nozzle outlet end is at least one round or non-round opening 2 to 10 microns deep and 5 to 15 microns wide, the depth preferably being 4.5 to 6.5 microns and the length 7 to 9 microns.

In the event of a plurality of nozzle openings, two being preferred, the directions of the jets from the nozzles in the nozzle member run parallel or slope relative to one another in the direction of the nozzle opening. In the case of a nozzle member with at least two nozzle openings at the inlet end the directions of the jets may be inclined relative to one another at an angle of 20 degrees to 160 degrees, preferably 60 to 150 degrees, most preferably 80 to 100°.

The nozzle openings are preferably arranged at a spacing of 10 to 200 microns, more preferably at a spacing of 10 to 100 microns, most preferably 30 to 70 microns. A spacing of 50 microns is most preferred. The directions of the jets accordingly meet in the vicinity of the nozzle openings.

The liquid pharmaceutical preparation meets the nozzle member at an entry pressure of up to 600 bar, preferably 200 to 300 bar, and is atomised by means of the nozzle openings to form an inhalable aerosol. The preferred particle or droplet sizes of the aerosol are up to 20 microns, preferably 3 to 10 microns.

The locking clamp contains a spring, preferably a cylindrical helical compression spring, as a store for mechanical energy. The spring acts on the power take-off flange as spring member, the movement of which is determined by the position of a locking member. The travel of the power take-off flange is precisely limited by an upper and lower stop. The spring is preferably tensioned by a force-transmitting gear, e.g. a helical thrust gear, by means of an external torque which is produced by rotating the upper housing part relative to the spring housing in the lower housing part. In this case, the upper housing part and the power take-off flange contain a single or multiple spline gear.

The locking member with engaging locking surfaces is annually disposed about the power take-off flange. It consists, for example, of an inherently radially elastically deformable ring made of plastics or metal. The ring is disposed in a plane at right angles to the atomiser axis. After the tensioning of the spring, the locking surfaces of the locking member move into the path of the power take-off flange and prevent the spring from being released. The locking member is actuated by a button. The actuating button is connected or coupled to the locking member. In order to actuate the locking clamp, the actuating button is pushed parallel to the ring plane, preferably into the atomiser; at the same time the deformable ring is deformed in the ring plane. Details of the construction of the locking clamp are described in WO 97/20590.

The lower housing part is pushed axially over the spring housing and covers the mounting, the drive of the spindle and the storage container for the fluid.

When the atomiser is actuated, the upper housing part is rotated relative to the lower housing part, the latter carrying the spring housing with it. The spring is compressed and tensioned by means of the helical thrust gear and the locking mechanism engages automatically. The angle of rotation is preferably a whole-number fraction of 360 degrees, e.g. 180 degrees. At the same time as the spring is tensioned, the power take-off component is pushed a certain distance in the upper housing part, the hollow piston is pulled back within the cylinder in the pump housing, as a result of which some of the fluid is sucked out of the storage container into the high pressure chamber in front of the nozzle.

A plurality of exchangeable storage containers containing the fluid to be atomised can be pushed into the atomiser as required and then used. The storage container contains the aqueous aerosol preparation according to the invention.

The atomising process is initiated by gently pressing the actuating button. The locking mechanism then opens the way for the power take-off component. The tensioned spring pushes the piston into the cylinder of the pump housing. The fluid leaves the nozzle of the atomiser in atomised form.

Other details of construction are disclosed in PCT applications WO 97/12683 and WO 97/20590, to which reference is hereby made.

The components of the atomiser (nebuliser) are made from a material suitable for their purpose. The housing of the atomiser and—if operation permits—other components too are preferably made of plastics, e.g. by injection moulding. For medical purposes, physiologically harmless materials are used.

FIGS. 1a/b, which are identical to FIG. 6a/b of WO 97/12687, illustrate the nebuliser (Respimat®) with which the aqueous aerosol preparations according to the invention can advantageously be inhaled.

FIG. 1A shows a longitudinal section through the atomiser with the spring under tension. FIG. 1B shows a longitudinal section through the atomiser with the spring released.

The upper housing part (51) contains the pump housing (52), at whose end is mounted the holder (53) for the atomiser nozzle. In the holder is the nozzle member (54) and a filter (55). The hollow piston (57) secured in the power take-off flange (56) of the locking clamp projects partially into the cylinder of the pump housing. At its end the hollow piston carries the valve member (58). The hollow piston is sealed off by means of the gasket (59). Inside the upper housing part is the stop (60) on which the power take-off flange abuts when the spring is released. On the power take-off flange is the stop (61) on which the power take-off flange abuts when the spring is tensioned. After the spring has been tensioned, the locking member (62) moves between the stop (61) and a support (63) in the upper housing part. The actuating button (64) is connected to the locking member. The upper housing part ends in the mouthpiece (65) and is sealed off by means of the protective cap (66) which can be fitted thereon.

The spring housing (67) with compression spring (68) is rotatably mounted on the upper housing part by means of the snap-fit lugs (69) and rotary bearing. The lower housing part (70) is pushed over the spring housing. Inside the spring housing is the replaceable storage container (71) for the fluid which is to be atomised (72). The storage container is sealed off by means of the stopper (73) through which the hollow piston projects into the storage container and dips its end in the fluid (store of active substance solution).

The spindle (74) for the mechanical counter is mounted in the casing surface of the spring housing. The drive pinion (75) is located on the end of the spindle which faces the upper housing part. The slider (76) is located on the spindle.

The nebuliser described above is suitable for nebulising the aerosol preparations according to the invention to form an aerosol suitable for inhalation.

If the formulation according to the invention is nebulised by the technology described above (Respimat®), the mass delivered should correspond to a defined amount with a tolerance range of not more than 25%, preferably 20% of this amount, in at least 97%, preferably at least 98% of all actuations (sprays) of the inhaler. Preferably, between 5 and 30 mg, particularly preferably between 5 and 20 mg of formulation, are delivered per spray as a defined mass.

However, the formulation according to the invention may also be nebulised by means of inhalers other than those described above, e.g. jet-stream inhalers.

Accordingly, in another aspect, the present invention relates to pharmaceutical compositions in the form of propellant-free inhalable solutions as described above in conjunction with a device suitable for administering these solutions, preferably in conjunction with the Respimat®. The present invention is preferably directed to propellant-free inhalable solutions characterised by the active substance combination of 1 and 2 according to the invention in conjunction with the device known by the name Respimat®. Moreover, the present invention relates to the inhalation devices mentioned above, preferably the Respimat®, characterised in that they contain the propellant-free inhalable solutions according to the invention described hereinbefore.

The propellant-free solutions for inhalation according to the invention may also be presented as concentrates or sterile ready-to-use solutions for inhalation, in addition to the abovementioned solutions intended for administration using the Respimat. Ready-to-use solutions for inhalation may be produced from the concentrates, for example by the addition of isotonic saline solutions. Sterile ready-to-use solutions for inhalation can be administered using energy-operated free-standing or portable nebulisers which produce inhalable aerosols by ultrasound or compressed air by the venturi principle or other principles.

Accordingly, in another aspect the present invention relates to pharmaceutical compositions in the form of propellant-free inhalable solutions as described above, which take the form of concentrates or sterile ready-to-use solutions, in conjunction with a device suitable for administering these solutions, characterised in that this device is an energy-operated free-standing or portable nebuliser which produces inhalable aerosols by ultrasound or compressed air by the venturi principle or other principles.

The following Examples serve to illustrate the present invention in more detail, although without restricting the scope of the invention to the following embodiments provided by way of example.

Starting Materials

Tiotropium Bromide:

The tiotropium bromide used in the following examples of formulations may be obtained as described in European Patent Application EP 418 716 A1.

The inhalable powders according to the invention may also be prepared using crystalline tiotropium bromide monohydrate. This crystalline tiotropium bromide monohydrate may be obtained by the following method.

15.0 kg of tiotropium bromide are added to 25.7 kg of water in a suitable reaction vessel. The mixture is heated to 80-90° C. and stirred at constant temperature until a clear solution is formed. Activated charcoal (0.8 kg), moistened with water, is suspended in 4.4 kg of water, this mixture is added to the tiotropium bromide-containing solution and rinsed with 4.3 kg of water. The mixture thus obtained is stirred for at least 15 min at 80-90° C. and then filtered through a heated filter into an apparatus which has been preheated to an outer temperature of 70° C. The filter is rinsed with 8.6 kg of water. The contents of the apparatus are cooled at 3-5° C. per 20 minutes to a temperature of 20-25° C. The apparatus is further cooled to 10-15° C. using cold water and crystallisation is completed by stirring for at least one hour. The crystals are isolated using a suction drier, the isolated crystal slurry is washed with 9 litres of cold water (10-15° C.) and cold acetone (10-15° C.). The crystals obtained are dried at 25° C. for 2 hours in a nitrogen current.

Yield: 13.4 kg of tiotropium bromide monohydrate (86% of theory)

The crystalline tiotropium bromide monohydrate thus obtained is micronised by known methods in order to prepare the active substance in the form of the average particle size which corresponds to the specifications according to the invention.

Salmeterol×½H₂SO₄:

Where reference is made to salmeterol×½H₂SO₄ in the following embodiments, this was obtained as follows:

A suspension of 2.5 g (4.15 mmol) of salmeterol xinafoate is dissolved in 6 ml of ethanol. A solution of 0.14 ml of 98% sulphuric acid is slowly added to the suspension with stirring. It is heated to 35-40° C. until fully dissolved. Then the solution is diluted with 10 ml of diethylether and inoculated with salmeterol sulphate. The salmeterol sulphate is suction filtered after 1.5 hours and washed with 20 ml of cold ethanol, acetone and diethylether. 1.5 g (78%) of salmeterol-½-sulphate are obtained.

Examples of Formulations

A) Powders for Inhalation:

1)

| Ingredients | μg per capsule |
|---|---|
| tiotropium bromide | 10.8 |
| salmeterol + ½ H₂SO₄ | 27.9 |
| lactose | 4961.3 |
| Total | 5000 |

2)

| Ingredients | μg per capsule |
|---|---|
| tiotropium bromide | 21.7 |
| salmeterol × ½ H₂SO₄ | 55.9 |
| lactose | 4922.4 |
| Total | 5000 |

3)

| Ingredients | μg per capsule |
|---|---|
| tiotropium bromide × H₂O | 22.5 |
| salmeterol × ½ H₂SO₄ | 55.9 |
| lactose | 4921.6 |
| Total | 5000 |

B) Inhalable Aerosols Containing Propellants:

1) Suspension Aerosol:

| Ingredients | % by weight |
|---|---|
| tiotropium bromide | 0.015 |
| salmeterol × ½ H₂SO₄ | 0.066 |
| soya lecithin | 0.2 |
| TG 11:TG12 = 2:3 | ad 100 |

2) Suspension Aerosol:

| Ingredients | % by weight |
|---|---|
| tiotropium bromide | 0.029 |
| salmeterol × ½ H₂SO₄ | 0.033 |
| absolute ethanol | 0.5 |
| isopropyl myristate | 0.1 |
| TG 227 | ad 100 |

3) Solution Aerosol:

| Ingredients | % by weight |
|---|---|
| tiotropium bromide | 0.042 |
| salmeterol × ½ H₂SO₄ | 0.047 |
| absolute ethanol | 30 |
| purified water | 1.5 |
| anhydrous citric acid | 0.002 |
| TG 134a | ad 100 |

C) Propellant-Free Inhalable Solutions:

1) Solution for Use in the Respimat®:

| Ingredients | mg/100 mL |
|---|---|
| tiotropium bromide | 148.5 |
| salmeterol × ½ H₂SO₄ | 276.7 |
| benzalkonium chloride | 10 |
| sodium edetate | 10 |
| hydrochloric acid (aq) | ad pH 2.9 |
| water | ad 100 mL |

Use of the solution in the Respimat leads to a dosage of 10 μg per dose of 1 and 25 μg/dose of 2.

2) Solution for Use in the Respimat®:

| Ingredients | mg/100 mL |
|---|---|
| tiotropium bromide | 148.5 |
| salmeterol × ½ H₂SO₄ | 276.7 |
| benzalkonium chloride | 10 |
| hydrochloric acid (aq) | ad pH 2.9 |
| water | ad 100 mL |

Use of the solution in the Respimat leads to a dosage of 10 μg per dose of 1 and 25 μg/dose of 2.

3) Solution for Use in the Respimat®:

| Ingredients | mg/100 mL |
|---|---|
| tiotropium bromide | 297.1 |
| salmeterol × ½ H₂SO₄ | 276.7 |
| benzalkonium chloride | 10 |
| sodium edetate | 10 |
| hydrochloric acid (aq) | ad pH 2.9 |
| water | ad 100 mL |

Use of the solution in the Respimat leads to a dosage of 20 μg per dose of 1 and 25 μg/dose of 2.

4) Solution for Use in the Respimat®:

| Ingredients | mg/100 mL |
|---|---|
| tiotropium bromide | 297.1 |
| salmeterol × ½ H₂SO₄ | 276.7 |
| benzalkonium chloride | 10 |
| hydrochloric acid (aq) | ad pH 2.9 |
| water | ad 100 mL |

Use of the solution in the Respimat leads to a dosage of 20 μg per dose of 1 and 25 μg/dose of 2.

5) Solution for Use in the Respimat®:

| Ingredients | mg/100 mL |
|---|---|
| tiotropium bromide | 148.5 |
| salmeterol × ½ H₂SO₄ | 1106.3 |
| benzalkonium chloride | 8 |
| sodium edetate | 50 |
| hydrochloric acid (aq) | ad pH 2.5 |
| water | ad 100 mL |

Use of the solution in the Respimat leads to a dosage of 10 μg per dose of 1 and 100 μg/dose of 2.

6) Solution for Use in the Respimat®:

| Ingredients | mg/100 mL |
|---|---|
| tiotropium bromide | 1.5 |
| salmeterol × ½ H₂SO₄ | 276.7 |
| benzalkonium chloride | 8 |
| sodium edetate | 10 |
| hydrochloric acid (aq) | ad pH 2.5 |
| water | ad 100 mL |

Use of the solution in the Respimat leads to a dosage of 0.1 μg per dose of 1 and 25 μg/dose of 2.

7) Solution for use in the Respimat®:

| Ingredients | mg/100 mL |
|---|---|
| tiotropium bromide | 14.9 |
| salmeterol × ½ H₂SO₄ | 1106.32 |
| benzalkonium chloride | 10 |
| sodium edetate | 50 |
| hydrochloric acid (aq) | ad pH 3.5 |
| water | ad 100 mL |

Use of the solution in the Respimat leads to a dosage of 1 μg per dose of 1 and 100 μg/dose of 2.

8) Solution for Use in the Respimat®:

| Ingredients | mg/100 mL |
|---|---|
| tiotropium bromide | 1486.1 |
| salmeterol × ½ H₂SO₄ | 1106.32 |
| benzalkonium chloride | 10 |
| sodium edetate | 10 |
| hydrochloric acid (aq) | ad pH 3.5 |
| water | ad 100 mL |

Use of the solution in the Respimat leads to a dosage of 100 μg per dose of 1 and 100 μg/dose of 2.

9) Concentrated Solution:

| Ingredients | mg/100 mL |
|---|---|
| tiotropium bromide | 1486.1 |
| salmeterol × ½ H₂SO₄ | 11063.2 |
| benzalkonium chloride | 20 |
| sodium edetate | 100 |
| hydrochloric acid (aq) | ad pH 3.5 |
| water | ad 100 mL |

We claim:

1. A pharmaceutical composition, comprising:
(a) a tiotropium salt (1); and
(b) a salmeterol salt (2),
optionally in the form of the enantiomers, mixtures of enantiomers, or in the form of the racemates thereof, optionally in the form of the solvates or hydrates and optionally together with a pharmaceutically acceptable excipient.

2. The pharmaceutical composition according to claim 1, wherein the tiotropium salt (1) and the salmeterol salt (2) are contained in a single preparation.

3. The pharmaceutical composition according to claim 1, wherein the tiotropium salt (1) and the salmeterol salt (2) are contained in two separate preparations.

4. The pharmaceutical composition according to claim 1, wherein the tiotropium salt (1) is a chloride, bromide, iodide, methanesulfonate, para-toluenesulfonate, or methylsulfate salt.

5. The pharmaceutical composition according to claim 1, wherein the salmeterol salt (2) is a chloride, bromide, sulfate, phosphate, methanesulfonate, acetate, fumarate, succinate, lactate, citrate, xinafoate, or maleate salt, with the proviso that the salmeterol salt (2) is not salmeterol xinafoate if the tiotropium salt (1) is tiotropium bromide.

6. The pharmaceutical composition according to claim 4, wherein the salmeterol salt (2) is a chloride, bromide, sulfate, phosphate, and methanesulfonate salts.

7. The pharmaceutical composition according to claim 1, wherein the relative amount of the tiotropium salt (1) to the salmeterol salt (2) are in a range from 1:300 to 30:1 based upon the ratios by weight of tiotropium (1′) to salmeterol (2′).

8. The pharmaceutical composition according to claim 1, wherein the relative amount of the tiotropium salt (1) to the salmeterol salt (2) are in a range from 1:230 to 20:1 based upon the ratios by weight of tiotropium (1′) to salmeterol (2′).

9. The pharmaceutical composition according to claim 1, wherein a single administration corresponds to a dosage of the active substance combination 1′ and 2′ of from 0.01 μg to 1000 μg.

10. The pharmaceutical composition according to claim 1, wherein a single administration corresponds to a dosage of the active substance combination 1′ and 2′ of from 0.01 μg to 200 μg.

11. The pharmaceutical composition according to claim 1, wherein the pharmaceutical composition is in a form suitable for inhalation administration.

12. The pharmaceutical composition according to claim 11, wherein the pharmaceutical composition is an inhalable powder.

13. The pharmaceutical composition according to claim 11, wherein the pharmaceutical composition is an inhalable aerosol containing propellant.

14. The pharmaceutical composition according to claim 11, wherein the pharmaceutical composition is an inhalable propellant-free solution.

15. The pharmaceutical composition according to claim 11, wherein the pharmaceutical composition is an inhalable powder, further comprising a suitable physiologically harmless adjuvant selected from monosaccharides, disaccharides, oligo- and polysaccharides, polyalcohols, salts, or mixtures thereof.

16. The inhalable powder according to claim 15, wherein the adjuvant has a maximum mean particle size of up to 250 μm.

17. The inhalable powder according to claim 16, wherein the adjuvant has a maximum mean particle size of between 10 μm and 150 μm.

18. A capsule containing the inhalable powder according to claim 15.

19. A capsule containing the inhalable powder according to claim 16.

20. A capsule containing the inhalable powder according to claim 17.

21. The inhalable powder according to claim 12, consisting essentially of the tiotropium salt (1) and the salmeterol salt (2).

22. The inhalable aerosol containing propellant according to claim 13, wherein the tiotropium salt (1) and the salmeterol salt (2) are in dissolved or dispersed form.

23. The inhalable aerosol containing propellant according to claim 22, wherein the propellant gas is a hydrocarbon or a halohydrocarbons.

24. The inhalable aerosol containing propellant according to claim 23, wherein the propellant gas is n-propane, n-butane, isobutane, or the chlorinated and/or fluorinated derivatives of methane, ethane, propane, butane, cyclopropane, or cyclobutane.

25. The inhalable aerosol containing propellant according to claim 22, wherein the propellant gas is TG11, TG12, TG134a, TG227, or a mixture thereof.

26. The inhalable aerosol containing propellant according to claim 22, further comprising a cosolvent, stabilizer, surfactant, antioxidant, lubricant, or agent for adjusting the pH.

27. The inhalable aerosol containing propellant according to claim 22, wherein the amount of the tiotropium salt (1) or the salmeterol salt (2) is up to 5% by weight of the pharmaceutical composition based upon the weight of tiotropium (1′) or salmeterol (2′), respectively.

28. The inhalable propellant-free solution according to claim 14, further comprising a solvent selected from water, ethanol, or a mixture of water and ethanol.

29. The inhalable propellant-free solution according to claim 28, wherein the pH of the solution is in the range of 2 to 7.

30. The inhalable propellant-free solution according to claim 29, wherein the pH of the solution is in the range of 2 to 5.

31. The inhalable propellant-free solution according to claim 29, wherein the pH is adjusted using an acid selected from hydrochloric acid, hydrobromic acid, nitric acid, sulfuric acid, ascorbic acid, citric acid, malic acid, tartaric acid, maleic acid, succinic acid, fumaric acid, acetic acid, formic acid, and propionic acid, or mixtures thereof.

32. The inhalable propellant-free solution according to claim 28, further comprising an additional cosolvent or adjuvant.

33. The inhalable propellant-free solution according to claim 32, wherein the additional cosolvent has one or more hydroxyl or other polar groups.

34. The inhalable propellant-free solution according to claim 33, wherein the additional cosolvent is an alcohol, glycol, glycerol, polyoxyethylene alcohol, or polyoxyethylene fatty acid ester.

35. The inhalable propellant-free solution according to claim 33, wherein the additional cosolvent is isopropyl alcohol, propylene glycol, polyethylene glycol, polypropylene glycol, glycol ether, glycerol, polyoxyethylene alcohol, or a polyoxyethylene fatty acid ester.

36. The inhalable propellant-free solution according to claim 32, wherein the adjuvant is selected from a surfactant, stabilizer, complexing agent, antioxidant, preservative, flavoring, pharmacologically harmless salt, or vitamin.

37. The inhalable propellant-free solution according to claim 32, wherein the adjuvant is edetic acid or a salt of edetic acid.

38. The inhalable propellant-free solution according to claim 32, wherein the adjuvant is ascorbic acid, vitamin A, vitamin E, or a tocopherols.

39. The inhalable propellant-free solution according to claim 32, wherein the adjuvant is cetylpyridinium chloride, benzalkonium chloride, benzoic acid, or a benzoate.

40. The inhalable propellant-free solution according to claim 14, consisting essentially of: the tiotropium salt (1), the salmeterol salt (2), benzalkonium chloride, sodium edetate, and the solvent.

41. The inhalable propellant-free solution according to claim 14, consisting essentially of: the tiotropium salt (1), the salmeterol salt (2), benzalkonium chloride, and the solvent.

42. The inhalable propellant-free solution according to claim 14, wherein the inhalable propellant-free solution is a concentrate or sterile ready-to-use solution for inhalation.

43. A method of administering the inhalable propellant-free solution according to claim 14 to a patient, comprising nebulizing the inhalable propellant-free solution in an inhaler according to WO 91/14468 or an inhaler as described in FIGS. 6a and 6b of WO 97/12687.

44. A method of administering the inhalable propellant-free solution according to claim 14 to a patient, comprising nebulizing the inhalable propellant-free solution in an energy-operated free-standing or portable nebulizer which produces inhalable aerosols by means of ultrasound or compressed air according to the Venturi principle or other principles.

45. A method of treating a respiratory disease, the method comprising administering to a patient in need thereof an effective amount of a pharmaceutical composition according to one of claims 1, 4, 5, 6, 7, 8, 9, or 10.

46. The method according to claim 45, wherein the respiratory disease is asthma or COPD.
